(19) United States
(12) Patent Application Publication
(10) Pub. No.: US 2008/0071379 A1
(43) Pub. Date: Mar. 20, 2008

(54) Intervertebral disc replacement

(76) Inventors: Mark Rydell, Golden Valley, MN (US); Steve Crosby, Prior Lake, MN (US); Britt Norton, Eden Prairie, MN (US); Jeffrey Felt, Excelsior, MN (US); Russ Felt, St. Paul, MN (US); Frank Cammisa JR., New York, NY (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/800,982

(22) Filed: May 8, 2007

Related U.S. Application Data
(60) Provisional application No. 60/799,245, filed on May 10, 2006.

(51) Int. Cl. A61F 2/44

(52) U.S. Cl. 623/17.16

(57) ABSTRACT

The present invention is an intervertebral disc replacement for replacing a disc between a first vertebra and a second vertebra including an upper containment structure adapted to contact a lower surface of a first vertebra, a lower containment structure adapted to contact an upper surface of a second vertebra, where the upper and lower containment structures are adapted to centrally contain a deformable support body positioned between the upper and lower containment structures.

### FIELD OF THE INVENTION

The present invention relates generally to an implantable artificial spinal disc device. More particularly, the present invention relates to an artificial disc for replacing a spinal disc.

### BACKGROUND OF THE INVENTION

The spinal motion segment consists of a unit of spinal anatomy bounded by two vertebral bodies, including the two vertebral bodies, the interposed intervertebral disc, as well as the attached ligaments, muscles, and the facet joints. The disc consists of the end plates at the surfaces of the vertebral bones, the soft inner core, called the nucleus pulposus and the annulus fibrosus ligament that circumferentially surrounds the nucleus and connects the vertebrae together. In normal discs, the nucleus cushions applied loads, thus protecting the other elements of the spinal motion segment. The nucleus in a normal disc responds to compression forces by bulging outward against the vertebral end plates and the annulus fibrosus. The annulus consists of collagen fibers and a smaller amount of elastic fibers, both of which are effective in resisting tension forces. However, the annulus on its own is not very effective in withstanding compression and shear forces.

As people age the intervertebral discs often degenerate naturally. Degeneration of the intervertebral discs may also occur in people as a result of degenerative disc disease. Degenerative disc disease of the spine is one of the most common conditions causing back pain and disability in our population. When a disc degenerates, the nucleus dehydrates. When a nucleus dehydrates, its ability to act as a cushion is reduced. Because the dehydrated nucleus is no longer able to bear loads, the loads are transferred to the annulus and to the facet joints. The annulus and facet joints are not capable of withstanding their increased share of the applied compression and torsional loads, and as such, they gradually deteriorate. As the annulus and facet joints deteriorate, many other effects ensue, including the narrowing of the interspace, bony spur formation, fragmentation of the annulus, fracture and deterioration of the cartilaginous end plates, and deterioration of the cartilage of the facet joints. The annulus and facet joints lose their structural stability and subtle but pathologic motions occur between the spinal bones.

As the annulus loses stability it tends to bulge outward and may develop a tear allowing nucleus material to extrude. Breakdown products of the disc, including macroscopic debris, microscopic particles, and noxious biochemical substances build up. The particles and debris may produce sciatica and the noxious biochemical substances can irritate sensitive nerve endings in and around the disc and produce low back pain. Affected individuals experience muscle spasms, reduced flexibility of the low back, and pain when ordinary movements of the trunk are attempted.

Degeneration of a disc is irreversible. In some cases, the body will eventually stiffen the joints of the motion segment, effectively re-stabilizing the discs. Even in the cases where re-stabilization occurs, the process can take many years and patients often continue to experience disabling pain. Extended painful episodes of longer than three months often leads patients to seek a surgical solution for their pain.

Several methods have been devised to attempt to stabilize the spinal motion segment. Some of these methods include: applying rigid or semi-rigid support members on the sides of the motion segment; removing and replacing the entire disc with an articulating artificial device; removing and replacing the nucleus; and spinal fusion involving permanently fusing the vertebrae adjacent the affected disc.

Several artificial disc replacements exist. One such device is disclosed in U.S. Pat. No. 6,156,067 to Bryan et al. The endoprosthesis disclosed in the '067 Patent requires a rigid annular gasket portion that surrounds a supple core. The disc replacement disclosed in U.S. Pat. No. 6,966,929 to Mitchell requires an asymmetric spacer that mates with cavities in each of two plates. Another disc prosthesis is disclosed in U.S. Pat. No. 7,025,787 to Bryan et al. The prosthesis disclosed in the '787 Patent requires a sheath that encapsulates a central body. U.S. Pat. No. 7,060,100 to Ferree discloses an artificial disc and joint replacement that includes a fluid filled cushion attached to the endplate supports. The motion disc disclosed in U.S. Pat. No. 7,156,876 to Moumene et al includes a central articulating core surrounded by a shock absorbing component attached to support plates.

While many artificial discs exist, there remains a need for an artificial disc that mimics the natural movement of a healthy spinal joint.

### SUMMARY OF THE INVENTION

The implant of the present invention includes centrally oriented containment of a support body allowing for axial, rotational, and bending movements mimicking the movement of a healthy spinal joint. In an embodiment the implant of the present invention may include a support body positioned between an upper and lower containment structure.

According to one aspect of the present invention, the support body is comprised of a shock absorbing material. In one embodiment of the present invention, the shock absorbing material may be a polymer such as, for example, cross-linked thermoset polyurethane or other polymer having similar characteristics. In an embodiment of the present invention, the support body may be deformable in response to surface features on the containment structures.

According to one embodiment of the present invention upper and lower containment structures may be positioned between two adjacent vertebrae. According to one aspect of the present invention, the two adjacent vertebrae may be cervical vertebrae.

In one embodiment of the present invention, the containment structures may be comprised of a polymeric material. In another embodiment of the present invention, the containment structures may be comprised of metal, a metal alloy or other biocompatible material.

In an embodiment of the present invention, the containment structures may be affixed, attached, bonded, mated or otherwise engaged to the vertebral endplates. In one embodiment of the present invention, the support body may conform to the shape of the containment structures. In another embodiment, the support body may be affixed, attached, bonded, mated or otherwise engaged to the containment structures.

### BRIEF DESCRIPTION OF THE DRAWINGS

FIG. 1 depicts a side view of an embodiment of the present invention.

FIG. 2 depicts an end view of an embodiment of the present invention.

FIG. 3 depicts a side view of an embodiment of the present invention implanted between adjacent vertebrae.

FIG. 4 depicts an anterior view of an embodiment of the present invention.

FIG. 5 depicts an anterior three-dimensional view of an embodiment of the present invention.

FIG. 6 depicts an embodiment of the present invention including tab-like features.

FIGS. 7a and 7b depict an embodiment of the present invention including a circular and square shaped support body.

FIG. 8 depicts a wedge shaped embodiment of the present invention.

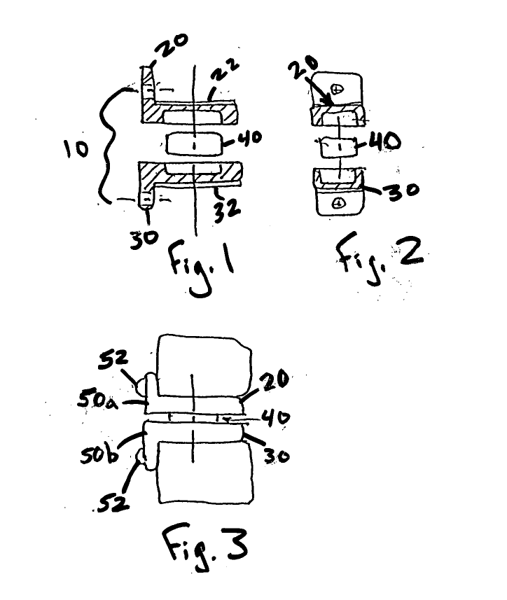

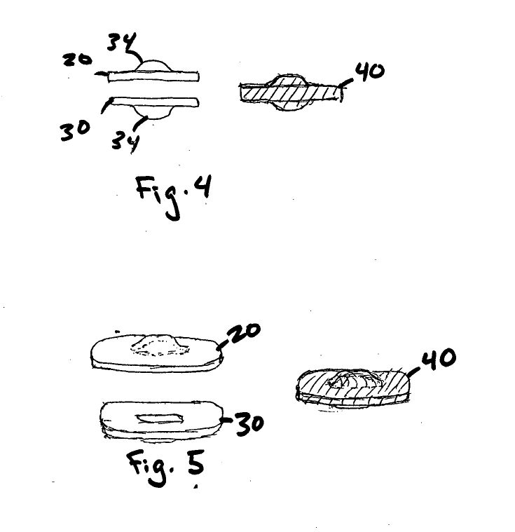

### DETAILED DESCRIPTION

As is depicted in FIG. 1, implant 10 of the present invention generally includes an upper containment structure 20, a lower containment structure 30 and a support body 40. Support body 40 may be comprised of a shock absorbing polymer of a compressive modulus similar to that of a healthy disc. In one embodiment of the present invention, support body 40 may be comprised of cross linked thermoset polyurethane. Any polymer of suitable hardness, strength, compressive modulus and biocompatibility to allow the artificial disc device to mimic the characteristics of a natural healthy disc may be used. In one embodiment, support body 40 may have a durometer in the range of about 35-95 Shore A or in the range of about 50-75 Shore D and a compressive modulus in the range of about 14-25 MPa. In one preferred embodiment, support body 40 is comprised of a softer material than containment structures 20 and 30.

Containment structures 20 and 30 may be comprised of a polymer of suitable hardness, strength, compressive modulus and biocompatibility, such as for example, polyetheretherketone (PEEK) or other suitable polymer. In an alternate embodiment, the containment structures 20 and 30 may be comprised of a porous form of PEEK having sufficient pore size to allow bony in-growth into the polymer to promote adhesion of the containment structures to the vertebral endplates. In an embodiment, containment structures 20 and 30 may have a hardness in the range of about 100 to 130 Rockwell R and a compressive modulus in the range of about 3000 to 7000 MPa. In an alternate embodiment, containment structures 20 and 30 may be comprised of solid or expanded forms of titanium, stainless steel, cobalt-chrome, other metals and/or alloys, ceramics, or any other suitable material.

In an embodiment according to the present invention, containment structures 20 and 30 may include features to affix, fasten, mate, bond or otherwise engage to the vertebral endplates. Examples of such features may include, but are not limited to: a keel or wedge shape 34 in either the anterior-posterior or medial-lateral direction or both directions in a T-shape or other multiaxial shape which fits into a corresponding indentation formed by the surgeon in the vertebrae; a textured surface to provide a matrix for bone in-growth which could be augmented with agents to enhance bone growth such as, for example, bone morphogenic proteins (BMP), transforming growth factors (TGF) and other osteoinductive materials and/or tabs 50 a and 50b which can be used to secure containment structures 20 and 30 to the anterior surface of the vertebral body using bone screws 52 or other suitable fastening components. In another embodiment of the present invention, containment structures 20 and 30 may include bone growth material that is laminated or otherwise attached on the vertebral end plate contact surfaces 22 and 32. In one embodiment upper and lower containment structures 20 and 30 may include tab-like features 50a and 50b that may be adapted to affix or otherwise connect the upper and lower containment structures to the outside of the vertebrae. In one embodiment, the tab-like features 50a and 50b may be laterally offset relative to each other, such that the tabs of one disc replacement will not interfere with another disc replacement used in an adjacent disc as shown in FIG. 6.

In contrast to most conventional disc replacements which utilize a peripheral containment system to contain the central nucleus-like component, implant 10 may include a centrally oriented containment system. Support body 40 may conform to the shape of containment structures 20 and 30 securing support body between containment structures 20 and 30. In an embodiment according to the present invention, support body 40 may include protrusions or other structures which fit into corresponding indentations or other structures on and/or in containment structures 20 and 30. Support body 40 may be deformable in response to various surface features of containment structures 20 and 30.

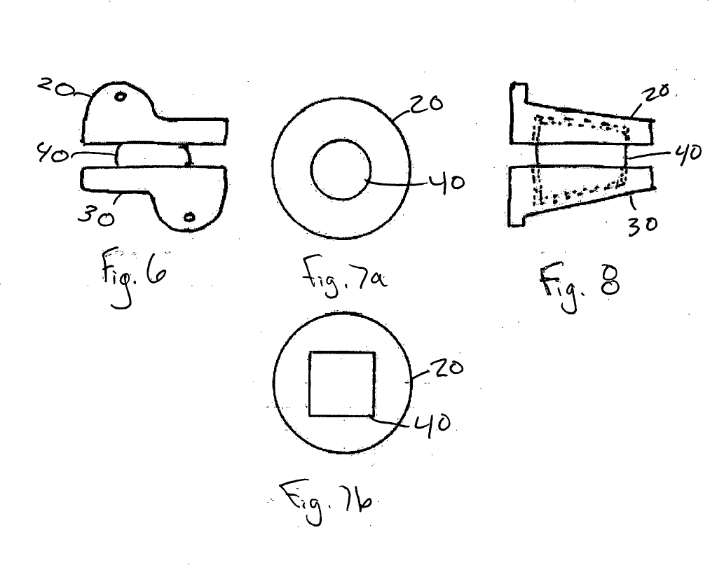

Support body 40 may be formed in different shapes. In an embodiment, support body 40 may be generally circular. As shown in FIG. 7a, a circular shape generally offers little to no resistance to limit torque. In another embodiment as shown in FIG. 7b, support body 40 may be configured in a generally square shape, “X” shape or any other shape that limits torque. In yet another embodiment, support body 40 may be in a generally wedge shape, such that the vertical height at the front of support body 40 is taller than the height at the back of support body 40. In one embodiment, as shown in FIG. 8 upper and lower containment structures 20 and 30 may be configured in a corresponding wedge shape. The wedge shape may allow for the same load to compress device 10 more in the front than in the back such that disc replacement 10 may be more flexible in flexion than in extension.

Containment structures 20 and 30 may entrap support body 40. In one embodiment of the present invention, containment structures 20 and 30 may include pockets, indentations or other structures that may entrap, constrain and/or contain support body 40. In one embodiment of the present invention, containment structures 20 and 30 may include posterior and/or anterior lips or other structures to further lock support body 40 into place. Containment structures 20 and 30 contain support body 40 without affecting the compressive ability of support body 40. The construction and arrangement of implant 10 allows axial, rotational, and bending movements such that the vertebral bodies may move in much the same manner as a healthy spinal joint.

In another embodiment, fibrous PEEK or any other suitable material may be wrapped around the periphery of the disc replacement mimicking a natural annulus. The fibrous PEEK may be woven into a fabric. In an embodiment, the weave may be woven off-bias, for example, at a 45 degree angle. This off-bias weave pattern mimics the weave pattern of a natural annulus. Placing fibrous PEEK about the periphery of the device may serve to limit rotational motion and limit flexion and extension in the same manner that a natural annulus limits motion.

In an embodiment of the present invention, upper containment structure 20 and lower containment structure 30 may be positioned in the interdiscal space prior to inserting support body 40. Once containment structures 20 and 30 are in position, support body 40 may then be positioned between containment structures 20 and 30. In an alternate embodiment of the present invention, support body 40 may be bonded, engaged or otherwise affixed between containment structures 20 and 30 prior to insertion. In this embodiment, implant 10 is inserted as a one piece composite disc replacement, which due to the compressive modulus of support body 40, allows movement in the joint space. In one preferred embodiment, implant 10 is inserted into a cervical disc space.

### Listing of Claims

A detailed listing of all claims that are, or were, in the present application, irrespective of whether the claim(s) remains under examination in the application are presented below. The claims are presented in ascending order and each includes one status identifier. Those claims not cancelled or withdrawn but amended by the current amendment utilize the following notations for amendment: 1. deleted matter is shown by strikethrough; and 2. added matter is shown by underlining.

1. An intervertebral disc replacement for replacing a disc between a first vertebra and a second vertebra comprising:
an upper containment structure adapted to contact a lower surface of a first vertebra;
a lower containment structure adapted to contact an upper surface of a second vertebra;
the upper and lower containment structures adapted to centrally contain a deformable support body positioned between the upper and lower containment structures.

2. The disc replacement of claim 1 wherein the upper and lower containment structures are comprised of a polymer having a hardness in the range of about 100 to 130 Rockwell R.

3. The disc replacement of claim 2 wherein the polymer is polyetheretherketone polymer.

4. The disc replacement of claim 3 wherein the polyetheretherketone polymer is porous having sufficient pore size to allow bone in-growth.

5. The disc replacement of claim 1 wherein the support body is comprised of a polymer having a durometer in the range of about 35 to 95 Shore A or 50 to 75 Shore D.

6. The disc replacement of claim 1 wherein the support body is deformable in response to surface features on the upper and lower containment structures.

7. The disc replacement of claim 1 wherein the support body conforms to the shape of the upper and lower containment structures.

8. The disc replacement of claim 1 wherein the support body has a compressive modulus in the range of about 14 to 25 MPa.

9. The disc replacement of claim 1 wherein the upper and lower containment structures have a compressive modulus in the range of about 3,000 to 7,000 MPa.

10. The disc replacement of claim 1 wherein the support body is attached to at least one of the upper and lower containment structures prior to insertion between the vertebrae.

11. The disc replacement of claim 1 wherein at least one of the upper and lower containment structures include a containment structure adapted to contain the support body.

12. The disc replacement of claim 1 wherein the support body may be shaped to limit torque.

13. The disc replacement of claim 1 wherein the support body is generally configured in a wedge shape and the containment structures are generally configured in a corresponding wedge shape.

14. The disc replacement of claim 1 further including fibrous PEEK adapted to be wrapped around the periphery of the disc replacement.
